(19) United States
(12) Patent Application Publication
(10) Pub. No.: US 2023/0373711 A1
(43) Pub. Date: Nov. 23, 2023

(54) WASTE DISPOSAL DEVICE AND FILM DISPENSING CASSETTE

(71) Applicant: ANGELCARE CANADA INC., Montreal (CA)

(72) Inventors: Michel MORAND, Verdun (CA); Hugo MOERMAN-ROUSSEAU, St-Hubert (CA)

(21) Appl. No.: 18/030,304

(22) PCT Filed: Oct. 5, 2021
(86) PCT No.: PCT/CA2021/051390

§ 371 Date: Apr. 5, 2023

Related U.S. Application Data
(60) Provisional application No. 63/087,562, filed on Oct. 5, 2020.

(51) Int. Cl. B65F 1/06; A61F 13/551; B65F 1/16; B65F 1/00

(52) U.S. Cl. CPC B65F 1/062; A61F 13/5512; A61F 13/5511; B65F 1/1646; B65F 1/0006; B65F 2210/1675; B65F 2240/132

(57) ABSTRACT

A waste disposal device may have a container having an opening at a top end, the container defining an inner cavity accessible through the opening. A bag twisting mechanism is adapted to hold a bag opened at the opening of the container with a closed end of the bag extending in the inner cavity, the bag twisting mechanism rotatably mounted to the container and rotatable reciprocally to twist the bag open or closed.

### CROSS-REFERENCE TO RELATED APPLICATION

The present application claims the priority of U.S. Patent Application No. 63/087,562, filed on Oct. 5, 2020, and incorporated herein by reference.

### FIELD OF THE APPLICATION

The present application relates to a film dispensing cassette of the type used with a waste disposal device and, more particularly but not exclusively, to a film dispensing cassette and waste disposal device for the disposal of odorous waste, such as diapers and litter.

### BACKGROUND OF THE ART

Waste disposal devices having odor-concealing systems with film dispensing cassettes are well known. Such waste disposal devices typically comprise a bag that is closed off by given mechanisms, to conceal the odors within the bag. U.S. Pat. No. 6,817,164 (Mauffette et al.) and U.S. Pat. No. 7,406,814 (Morand) propose such odor-concealing systems. However, it is desired to provide a waste disposal device that is simple in construction, notably in the way it closes the bags to conceal the odors in the bag. It may also be desired to reduce the consumption of plastics in such systems.

### SUMMARY OF THE APPLICATION

It is therefore an aim of the present disclosure to provide a novel waste disposal device.

It is therefore an aim of the present disclosure to provide a novel film dispensing cassette for waste disposal device.

It is a further aim of the present disclosure to provide a novel assembly of film dispensing cassette and waste disposal device.

Therefore, in accordance with a first aspect of the present disclosure, there is a waste disposal device comprising: a container having an opening at a top end, the container defining an inner cavity accessible through the opening, and a bag twisting mechanism adapted to hold a bag opened at the opening of the container with a closed end of the bag extending in the inner cavity, the bag twisting mechanism rotatably mounted to the container and rotatable reciprocally to twist the bag open or closed.

Further in accordance with the first aspect, for example, the bag twisting mechanism has a rotor to which a top portion of the bag is attached, the rotor defining a central opening for the bag to pass therethrough.

Still further in accordance with the first aspect, for example, the rotor has a top periphery shaped for the top portion of the bag to be hooked thereon.

Still further in accordance with the first aspect, for example, the top periphery has an obround shape.

Still further in accordance with the first aspect, for example, the rotor has a tubular funnel portion.

Still further in accordance with the first aspect, for example, the tubular funnel portion defines a circular shape of the central opening at a bottom of the tubular funnel portion.

Still further in accordance with the first aspect, for example, the tubular funnel portion transitions from the circular shape at the bottom to a non-circular shape at an upper end of the tubular funnel portion.

Still further in accordance with the first aspect, for example, the rotor has a base portion forming a rotational joint with the container, around the opening.

Still further in accordance with the first aspect, for example, the base portion is slidingly mounted onto a hub defined at the opening of the container.

Still further in accordance with the first aspect, for example, a handling ring is mounted onto the rotor and rotates therewith.

Still further in accordance with the first aspect, for example, the handling ring has a handle for manually moving the bag twisting mechanism reciprocally to twist the bag open or closed.

Still further in accordance with the first aspect, for example, the handling ring defines a funnel aligned with the opening.

Still further in accordance with the first aspect, for example, the handling ring has webs projecting from corners thereof in alignment with corners of the rotor for holding the top portion of the bag captive.

Still further in accordance with the first aspect, for example, a motorization unit may be provided to actuate rotations of the bag twisting mechanism.

Still further in accordance with the first aspect, for example, the bag may be provided.

Still further in accordance with the first aspect, for example, the bag is in a roll.

Still further in accordance with the first aspect, for example, the roll is in a casing.

Still further in accordance with the first aspect, for example, the container includes a compartment for receiving the casing.

Still further in accordance with the first aspect, for example, the compartment is accessible via a door at an underside of the container.

Still further in accordance with the first aspect, for example, the bag twisting mechanism rotates reciprocally for at most 90 degrees.

In accordance with a second aspect of the present disclosure, there is provided a method for opening and closing a bag hanging in a waste disposal device, comprising: rotating a bag twisting mechanism in a first direction, the bag twisting mechanism holding a bag at an opening of the waste disposal device, whereby the bag is twisted shut; and rotating the bag twisting mechanism in a second direction opposite to the first direction, whereby the bag is twisted open.

Further in accordance with the second aspect, for example, a bottom of the bag may be held.

Still further in accordance with the second aspect, for example, holding the bottom of the bag includes holding the bottom of the bag via a roll of tubular film including a series of the bag interconnected

Still further in accordance with the second aspect, for example, rotating the bag twisting mechanism in the first direction and in the second direction includes rotating the bag twisting mechanism along at most 90 degrees in rotation.

In accordance with a third aspect of the present disclosure, there is provided a bag comprising a tubular body with a top open end and a bottom closed end, the tubular body having a larger open diameter at the top open end, the tubular body narrowing into a narrower open diameter adjacent to a bottom closed end, and the tubular body in a flattened two-fold condition having a pair of side edges extending from a top edge to a bottom edge, the top edge delimiting the top open end, the bottom edge delimiting the bottom closed end, wherein at least one weld seam causes the narrowing and defines a taper of the open diameter extending to the bottom closed end.

Further in accordance with the third aspect, for example, the at least one weld seam includes a pair of segments defining the taper and being mirror images of one another.

Still further in accordance with the first aspect, for example, the segments are arcuate.

Still further in accordance with the first aspect, for example, flaps adjacent to the segments, the flaps excluded from the open diameter.

In accordance with a fourth aspect of the present disclosure, there is provided a bag-dispensing cassette comprising: a roll of bags connected end to end, wherein the bags spiral from a free end to a center of the roll; and a casing enclosing the roll, the casing having an opening through which an end of the roll passes to exit the casing; wherein between 2% and 15% of the bags in the roll in the casing in a loosened condition starting from the free end, a remainder of the bags in roll in the casing being in a taut condition.

Further in accordance with the fourth aspect, for example, the bags in the roll are as described above, the bags being connected bottom to top.

### BRIEF DESCRIPTION OF THE DRAWINGS

FIG. 1 is an assembly view of a top portion of a waste disposal device in accordance with a variant of the present disclosure;

FIG. 2 is a perspective view of the waste disposal device of FIG. 1, with a lid assembly open;

FIG. 3 is a perspective view of the waste disposal device of FIG. 1, with a handling ring removed;

FIG. 4 is a top view of the waste disposal device of FIG. 1, with a bag opened to receive waste;

FIG. 5 is a top view of the waste disposal device of FIG. 4 with the handling ring removed;

FIG. 6 is a top view of the waste disposal device of FIG. 4, with the bag twisted shut;

FIG. 7 is a sectional side view of the waste disposal device of FIG. 1, with the bag opened to receive waste;

FIG. 8 is a sectional side view of the waste disposal device of FIG. 1, with the bag twisted shut;

FIG. 9 is a sectional side view of the waste disposal device of FIG. 1, with the bag twisted shut and the lid assembly closed;

FIG. 10 is a sectional side view of the waste disposal device of FIG. 1, with the bag being removed;

FIG. 11 is an underside view of the waste disposal device of FIG. 1, showing a film dispensing cassette loaded in the waste disposal device;

FIG. 12 is a perspective view of the film dispensing cassette, with film concealed therein, in accordance with another aspect of the present disclosure;

FIG. 13 is a perspective view of the film dispensing cassette, with tabs removed to access the film;

FIG. 14 is a perspective view of the film dispensing cassette, with the film pulled out;

FIG. 15 is a perspective view of the film dispensing cassette, with the film pulled out and unfolded;

FIG. 16 is a perspective view of an exemplary roll of bags that is used with the waste disposal device of FIG. 1;

FIG. 17 is a schematic view of one of the bags of FIG. 16, in a flattened four-fold condition; and

FIG. 18 is a schematic view of the film dispensing cassette, in which a portion of film is in a loosened condition.

### DESCRIPTION OF THE PREFERRED EMBODIMENTS

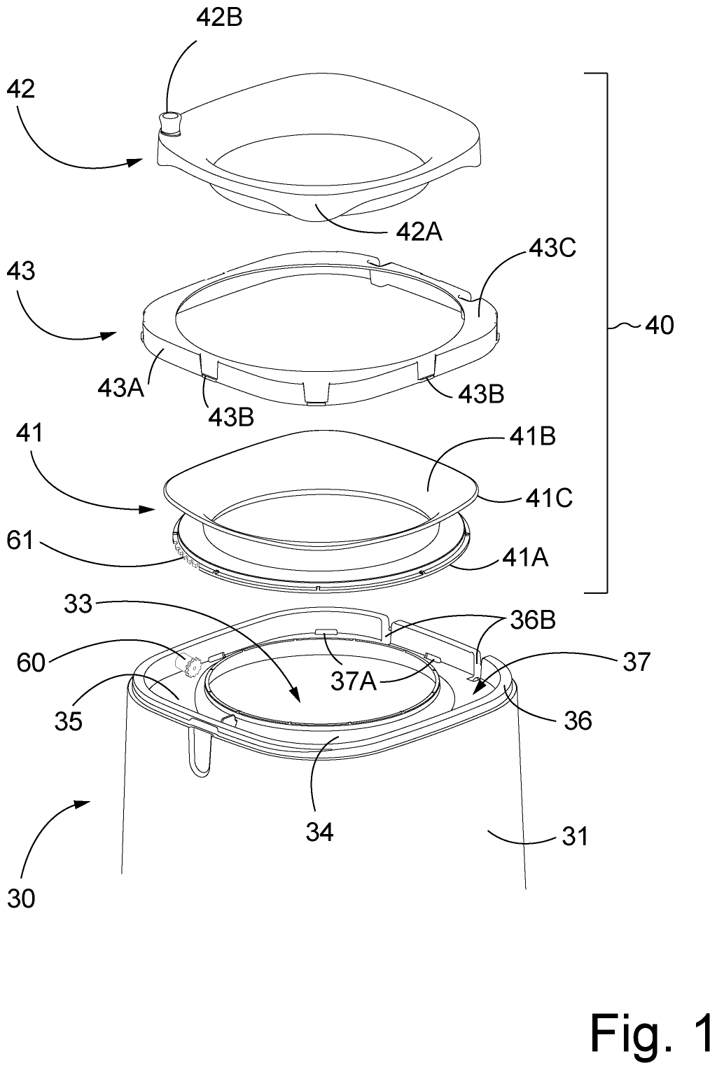

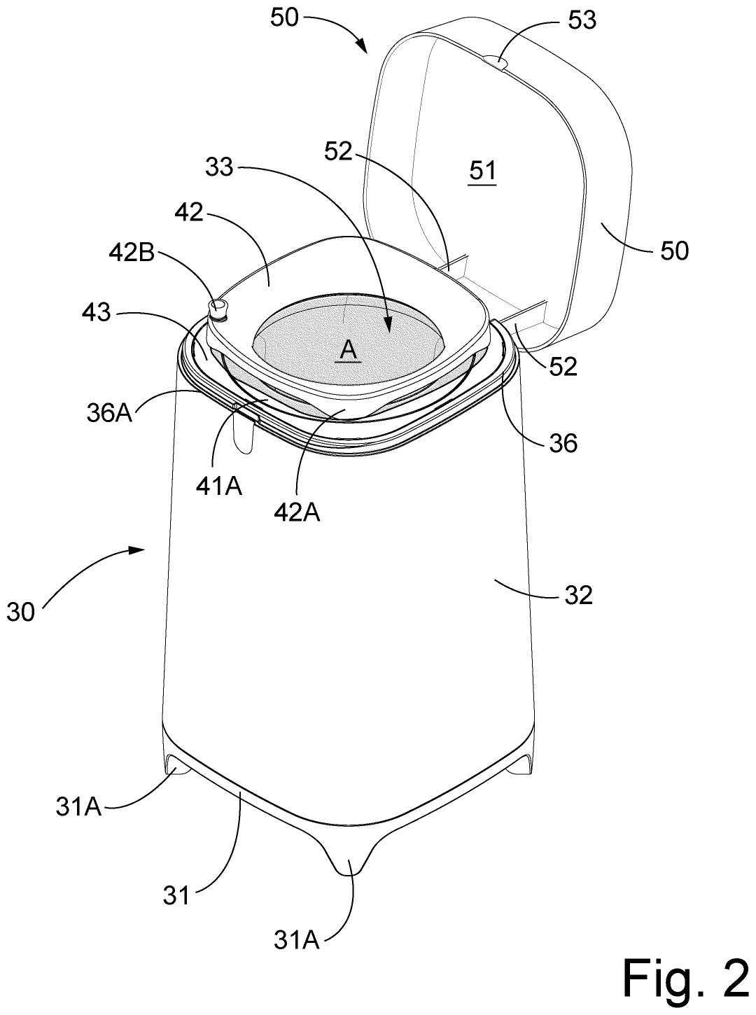

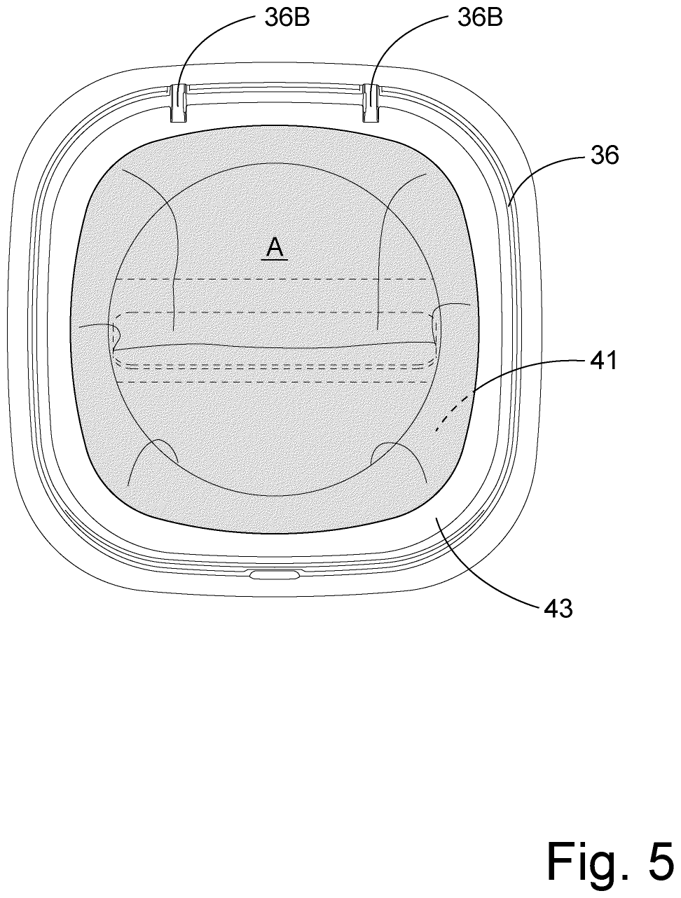

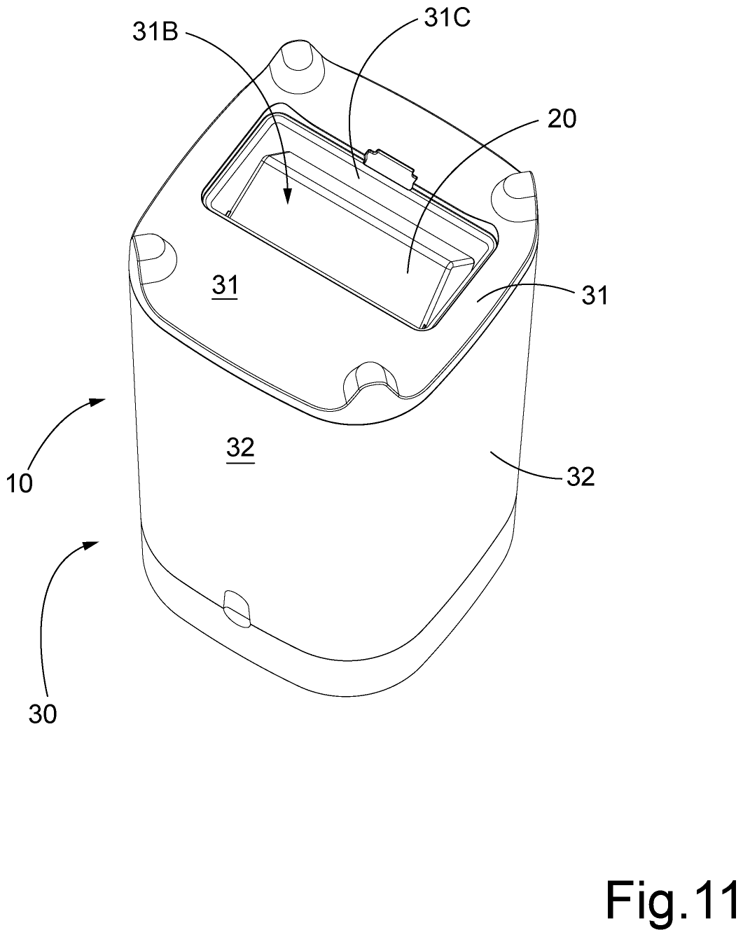

Referring to the drawings and more particularly to FIG. 1, a waste disposal device (hereinafter “device”) is generally shown at 10. The device 10 may be known as a garbage, a pail, a garbage pail, a trash, etc. The device 10 may be used with any suitable type of waste, including diapers, soiled litter, foodstuff, etc. The device 10 is of the type using a film dispensing cassette, one variant of which is shown at 20 (hereinafter “cassette” 20) dispensing film 25 that may form a tubular bag, generally shown at A in FIGS. 2 to 15. The film 25 may be a series of bags A connected end to end. Any other suitable type of bag dispenser may be used as well with the device 10. Moreover, the expression cassette is one among others to describe item 20, with other expressions including cartridge, refill, etc. The device 10 may have a container 30, a bag twisting mechanism 40, and/or a lid assembly 50. The film dispensing cassette 20 may be part of the device 10, or may also be an accessory used with the device 10, with the device 10 sold with or without the cassette 20. The cassette 20 may be used with other types of pails.

The container 30 forms the structure of the device 10, and accommodates and conceals the tubular bag containing waste. The container 30 is not limited to the shape illustrated in the Figs. Any suitable geometry is considered as well.

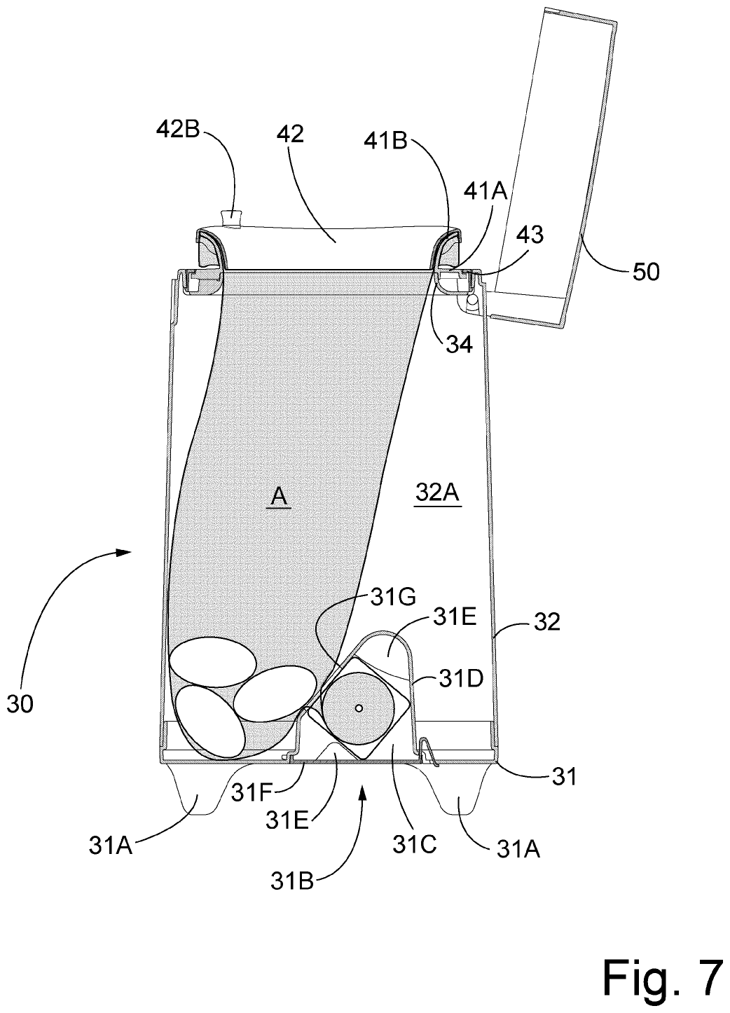

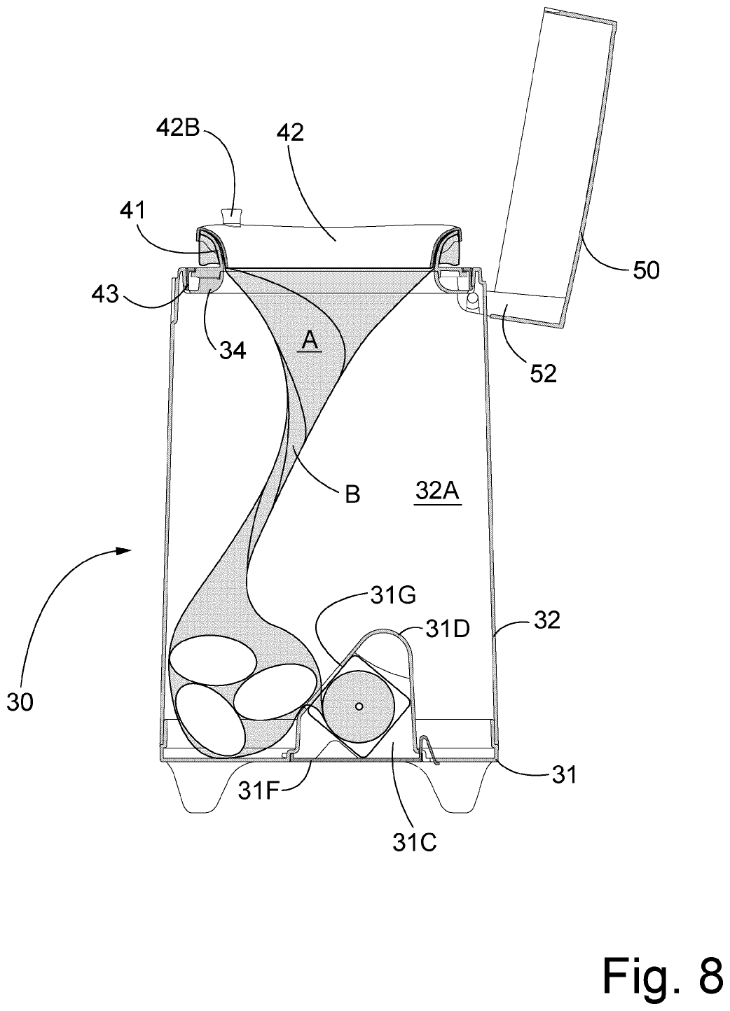

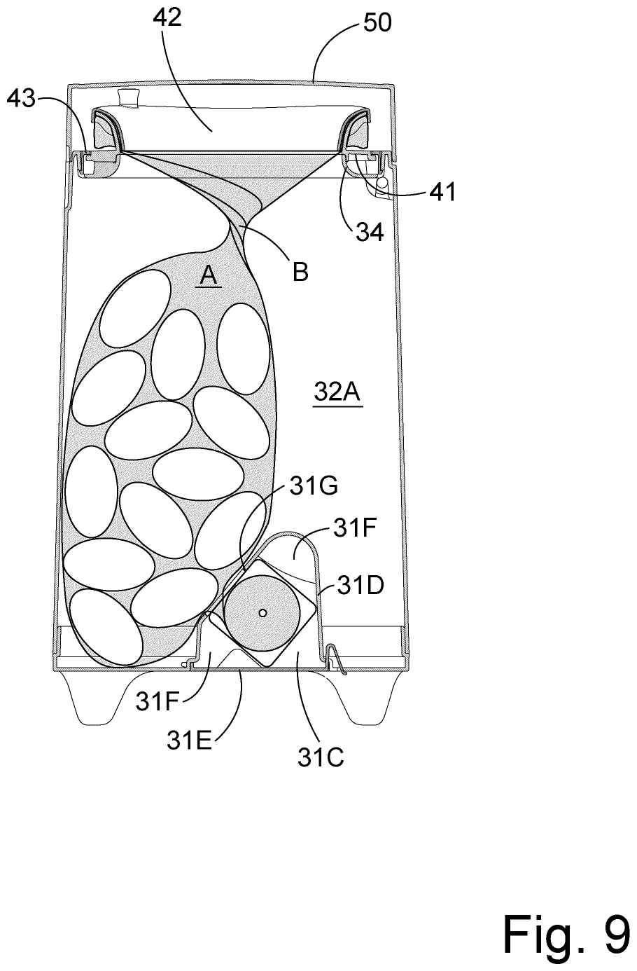

The bag twisting mechanism 40 serves as a rim from which the bag hangs open. The bag twisting mechanism 40 may be rotated to twist the bag A open and closed, for the bag to receive waste, and to then conceal the odors in the bag A, by forming a twist knot B. For example, FIG. 7 shows the bag A in its open condition, while FIGS. 8 and 9 show the bag A in its closed position, on itself.

The lid assembly 50 opens and closes a top opening of the container 30, through which the waste is thrown into the bag supported by the device 10.

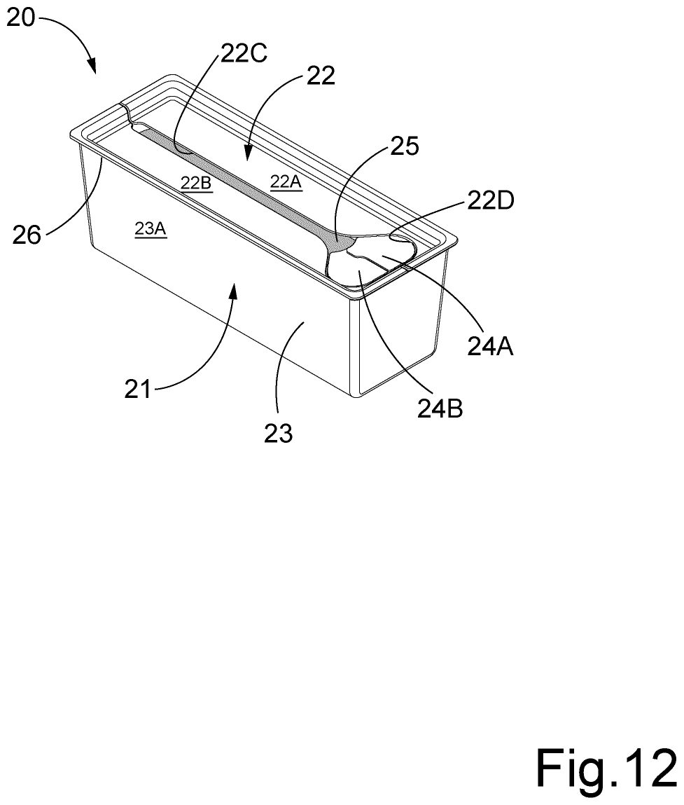

Referring to FIGS. 12 to 15, an exemplary embodiment of the cassette 20 is shown in greater details, with parts thereof shown in the 20s. The cassette 20 may be used in the device 10, but is not limited to being used with the device 10, as the cassette 20 may be used in other similar devices. For example, the cassette 20 may be used in devices such as shown in U.S. patent application Ser. No. 16/603,113, or in U.S. patent application Ser. No. 17/277,361, as examples among others.

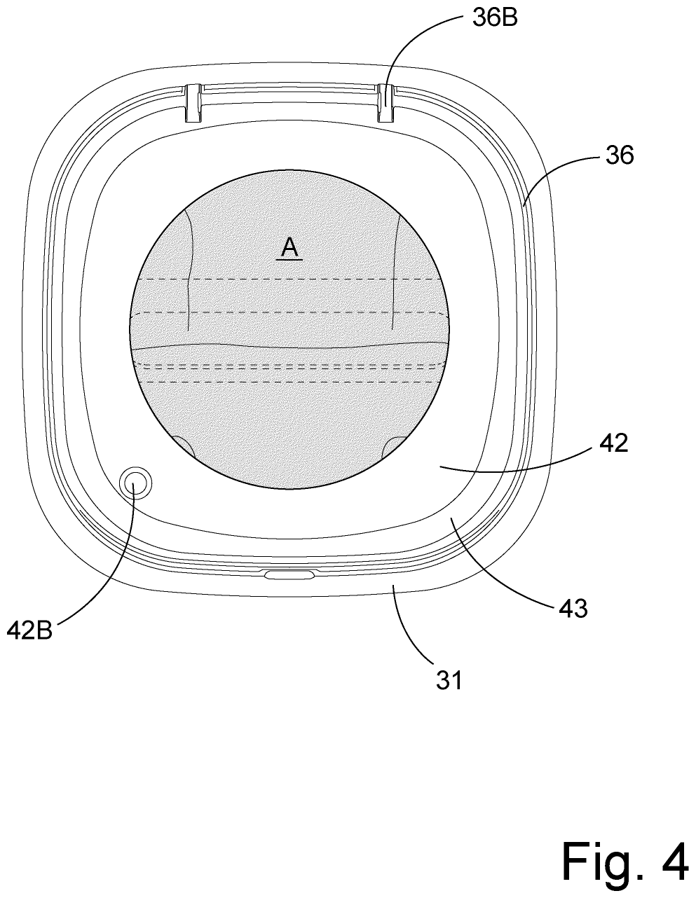

The cassette 20 has a casing 21 or body, having an elongated shape. The casing 21 may consist of one or more pieces interconnected to one another. In the illustrated embodiment, the casing 21 has a cover 22 and a receptacle 23. The cover 22 may be generally oriented sideways when used as described herein. However, in storage, shelving, the cassette 20 may be in any orientation, e.g., the cover 22 may be at the bottom while the receptacle 23 may be the top portion. In an embodiment, the cover 22 is made of a pair of panels 22A and 22B. The panels 22A and 22B may be flat as a possibility, as shown. One or both panels 22B may be concave from the exterior to define an inward abutment formation. The inward abutment formation may define an edge that catches a bag roll when film 25 is pulled out of the cassette 20, as detailed below. If a pair of panels 22A and 22B is present, a slit 22C (a.k.a., channel, elongated opening) may be defined between them for film to escape from an interior of the casing 21. The slit 22C may optionally flare into a larger portion to define an opening 22D covered by optional tear-off tabs or flaps 24A and 24B, for example large enough to allow one's fingers from picking a free end of the film 25. The opening 22D may not be present, in a variant. In an embodiment, the cover 22 may be an integrally formed monolithic piece as well, as an alternative to having the panels 22A and 22B. The tear-off tabs 24A and 24B may be integrally molded with the panels 22A and 22B, with tear lines to enable their breaking off. The tabs 24A and 24B may be a membrane (e.g., sticker) that can be pulled off, as well. In an embodiment, the panels 22A and 22B are molded with the receptacle 23 in such a way that living hinges are formed at the junction between the panels 22A and 22B and the receptacle 23. This may allow for film, such as in a roll, to be inserted in the inner cavity of the casing 21. The panels 22A and 22B may then be closed to the arrangement of FIG. 12, and fixed to the receptacle 23 (to block movement at the living hinges). Ultrasound welding, gluing, welding, soldering, are options to fix the panels 22A and 22B to the receptacle 23. As illustrated in FIG. 4, the film 25 may pass through the slit 22C, but it may also pass instead solely through the opening 22D. The cassette 20 may be with only one of the slit 22C and opening 22D, and not both. If there is no opening 22D to grasp the film 25, a free end thereof may be prearranged to project out of the casing 21 through the slit 22C.

Still referring to FIGS. 12-15 the receptacle 23 forms the inner cavity of the casing 21 by its hollow shape. The receptacle 23 has a wall 23A that forms most of the exposed peripheral surfacing of the cassette 20, with the cover 22, such as five walls in the illustrated variant. The receptacle 23 may define plain journal bearings, though not shown. In an embodiment, the film 25 is in a roll, with the roll accommodated in the receptacle 23. The expressions films, bags, tube, etc may be used to describe the roll 25. According to an embodiment, the roll 25 has bags made of tubular film connected end to end, with a free end of the roll 25 being defined by an open end of the bags. As seen hereinafter, the cassette 20 may be located in a bottom of the device 10, with the film of the roll 25 pulled upwardly and hung open to a top of the device 10, while remaining connected to a remainder of the roll 25.

The cassette 20 may have a rim 26. The rim 26 is formed at the periphery of the cassette 20, such as the top periphery when the cassette 20 is used. The rim 26 may consist of a portion of the cover 22 extending beyond the receptacle 23. Alternatively, the rim 26 may consist of a flange portion of the receptacle 23 extending beyond the cover 22. As shown in FIGS. 12 to 15, the rim 26 may be a combination of both a portion of the cover 22 and a flange at a bottom of the receptacle 23. The rim 26 may have numerous segments, continuous or discontinuous around the casing 21, though shown as being continuous. The rim 26 may also be known referred to as a shoulder(s), a flange, a lip, a strip, etc.

In an embodiment, the cassette 20 may include a component to temporarily close off the opening (e.g., in the absence of a door), such as a label, plastic film, wrapping, etc. For example, in-mold labelling (IML) material wraps the casing 21 covering at least a portion of the opening 22D and possibly the slit 22C. The IML material may have the predefined tear strip therein, in register with the opening 22D, for being torn off and allow access to the film 25. In an embodiment, the cover 22 and the receptacle 23 may be an integrally formed monolithic piece including the flap (if present). The IML material may be comolded with the monolithic piece, or may even be regarded as contributing to the monolithic nature as the IML material is integral with the cover 22 and the receptacle 23.

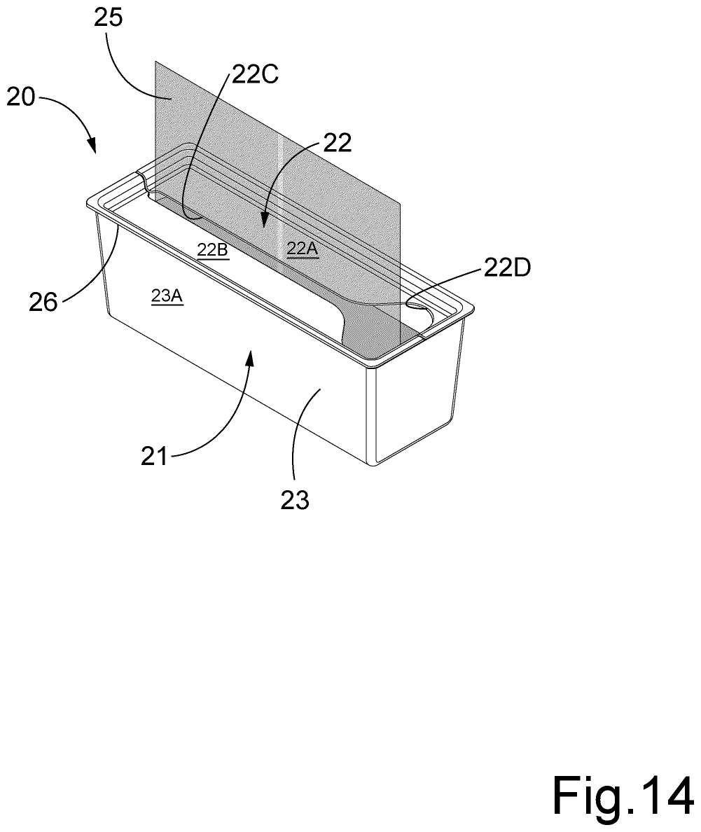

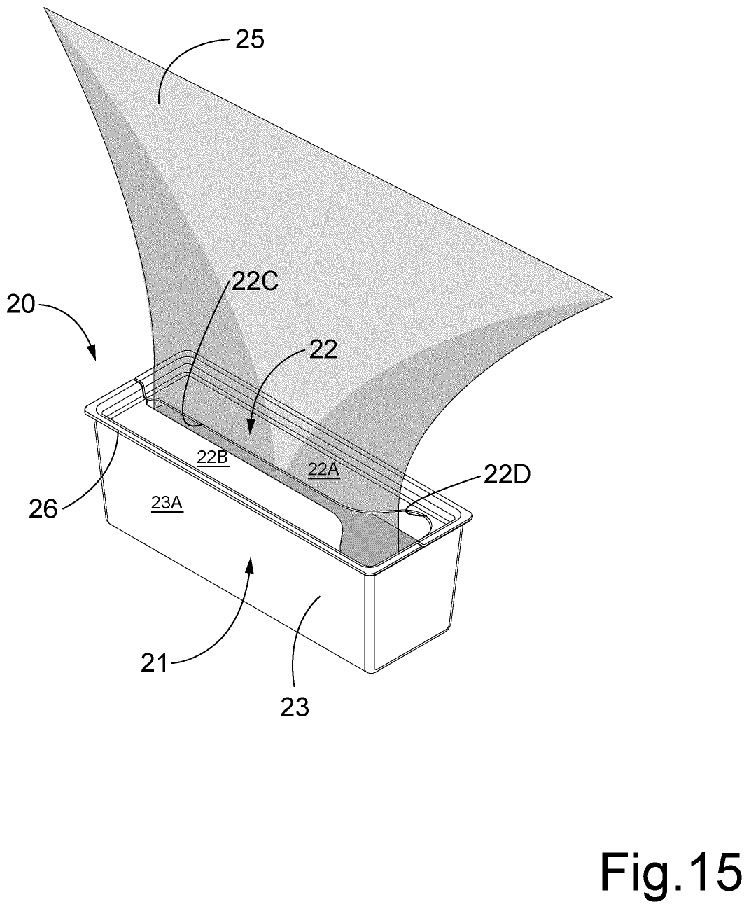

Now that the cassette 20 has been described, a deployment of the film from the cassette 20 is set forth. If the cassette 20 comes with the tabs 24A and/or 24B or with wrapping or like membrane (e.g., IML material) covering the opening 22D as in FIG. 12, the tabs 24A and/or 24B, wrapping or membrane are removed, to expose an interior of the cassette 20, and more particularly the film 25. The free end of film 25 may be connected (e.g., taped) to the tabs 24A and/or 24B, wrapping or membrane such that the free end of film 25 is pulled along. The free end of the film 25 may then be out of the casing 21, via slit 22C and/or opening 22D, in the manner shown in FIG. 14, such that the film may be pulled out of the casing 21 while the rest of the film 25 remains in the casing 21. As shown in FIGS. 14 and 15, the film 25 may be a folded roll of film, with two fold lines being present as an example. The free end of the film 25 is the open end.

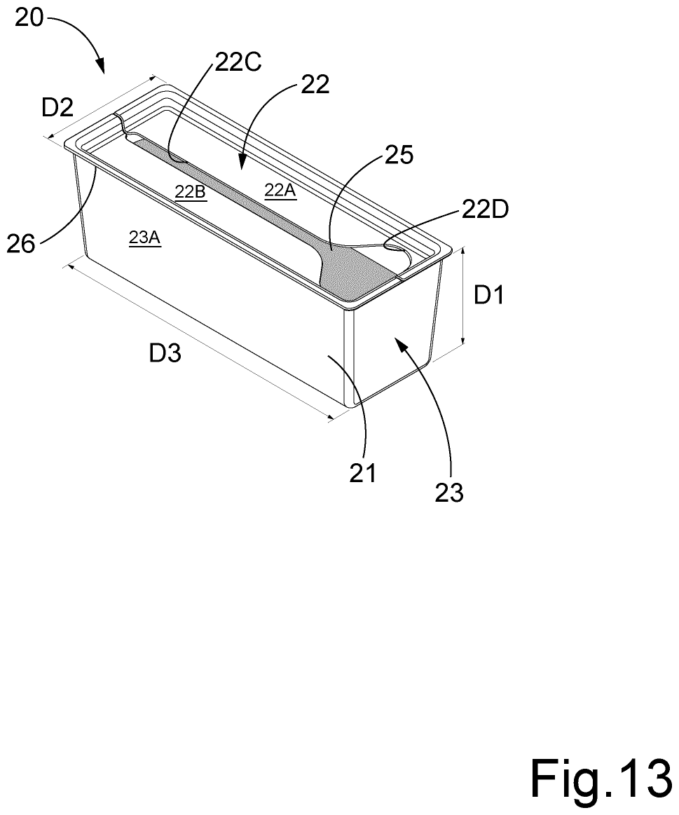

Referring to FIG. 13, exemplary dimensions are given. D1 may be between 5.0 cm and 7.0 cm. The cross-sectional shape of the casing 21 may be a trapezoid (excluding the rim 26), with the large base D2 of the trapezoid being between 5.0 cm and 7.0 cm. The smaller base of the trapezoid is between 4.5 cm and 5.5 cm. D3 may be between 14.0 cm and 18.0 cm, and may flare slightly toward the top for the casing 21 to be ejectable. The rim 26 may project by 2.0 to 6.0 mm. Other dimensions are possible, but these dimensions may be optimal in maximizing the amount of film 25 in the casing 21, from an environmental standpoint.

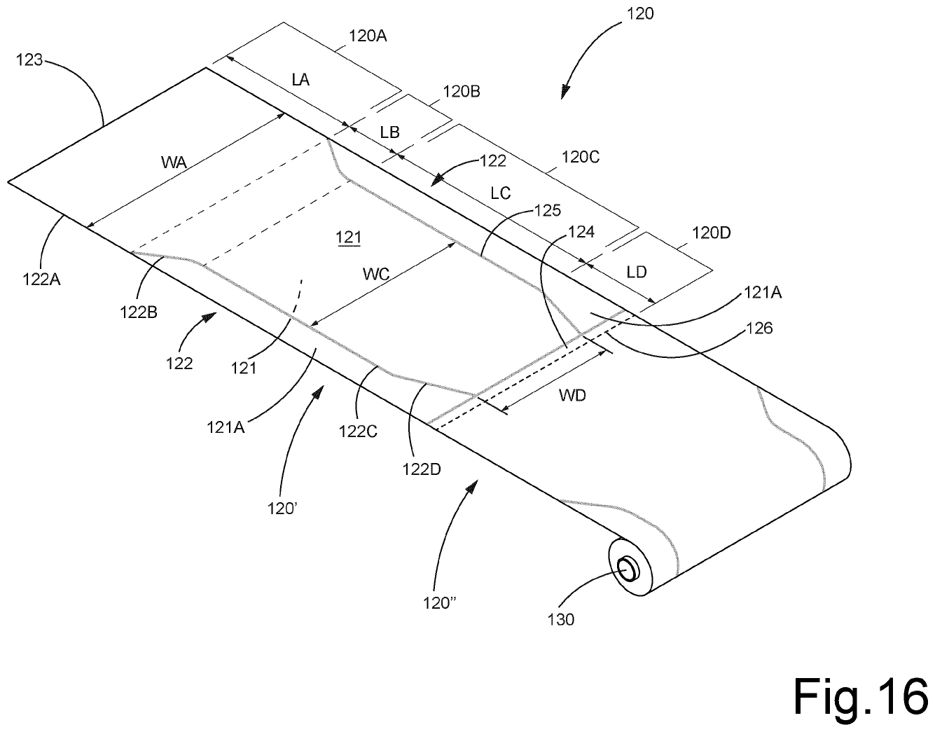

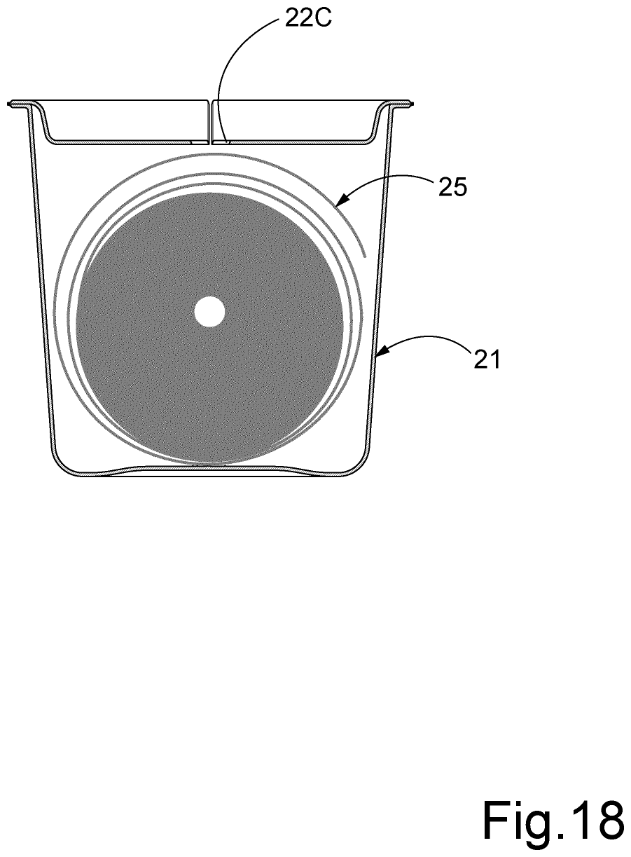

In order to facilitate the pulling out of the end of the roll of film 25 from the casing 21, via the slit 22C and/or opening 22D, an operation may be performed to loosen the roll to facilitate its manipulation. When the roll of film 25 is in a roll, it is in a taut condition, and may be installed in the casing 21. The roll of film 25 may be enclosed in the casing 21 with the cover 22 installed and shutting the opening of the casing 21 closed. In such a scenario the roll of film 25 remains in a taut condition (though not adhered to itself). The assembly of cassette 20 with roll of film 25 may then be rotated on itself, along a longitudinal axis that may correspond or be in proximity to and generally parallel to the central axis of the roll of film 25. However, the direction of rotation may be contrary to the direction of winding of the roll of film 25 on itself. Stated differently, if the roll of film 25 spirals towards its center in a clockwise direction, the assembly of the casing 21 and roll of film 25 is rotated counterclockwise. Because of friction between the roll of film 25 and the surfaces of the casing 21 and/or the different inertias of the roll of film 25 and of the casing 21, the outer passes of the roll of film 25 will loosen relative to the more central passes of the roll of film 25, as shown in FIG. 18. The outer passes of the roll of film 25 may even radially contact the surfaces of the casing 21. In an embodiment, between 2% and 15% of the end of the roll of film 25 achieves this loosened condition, while a remainder of the roll of film 25, forming an interior portion of the spiral of film, is in a taut condition. In a variant, the loosened condition may be described as a condition in which the film is not in a continuous contact with an inner pass of the roll of film 25. In a variant, the loosened condition may be described as a condition in which a tension in the film 25 is less than a tension in the portion of the film 25 that is taut. In a variant, the loosened condition of a portion of the film 25 is one in which the film 25 is not in a substantial cylindrical shape, in contrast to a portion of the film 25 in the taut condition. The film 25 may be as described in FIG. 16, but may have other configurations as well. For example the film 25 may be a flattened two-fold condition or in a flattened four-fold condition.

Referring to FIGS. 1 to 3, the container 30 may have a base portion 31 and a top portion 32, that may or may not be separable to provide access to an interior of the container 30. The container 30 may also be a single tubular unit. Other constructions are contemplated, for instance a tube or sleeve of fabric or paneling over structural beams, or a simple wire frame. The base portion 31 sits on the ground or floor when the device 10 is used, and has legs 31A for example, as shown, but with the legs 31A only being an option. An opening 31B opens into one or more compartments 31C that accommodates the cassette 20, as shown in FIGS. 7-11. The compartment 31C may form a frame 31D to receive and releasably support the cassette(s) 20. For example, a door 31E may open and close access to the opening, for the cassette 20 to be concealed into the compartment 31C. Abutments 31F or like spacers may be present in the frame 31D and/or in the door 31E to properly orient and immobilize the cassette 20 in the device 10. For example, the cassette 20 is shown having the slit 22C sideways, for the cassette 20 to apply friction onto the film 25. The compartment 31D may be accessible via an underside of the base portion 31 as shown, with a window 31G being present in the frame 31D to allow the film 25 to be deployed into the interior of the container 30.

The top portion 32 may have an upstanding tubular body that sits on the base portion 31. The top portion 32 defines an inner cavity 32A of the device 10, and hosts the bag A with waste. For instance, as shown, the top portion 32 sits on a shoulder defined by the base portion 31, to form a generally continuous outer surface of the container 30. Other arrangements are considered, and the base portion 31 and top portion 32 may be locked to one another (e.g., glued, bonded, etc).

Referring to FIG. 1, an opening 33 is at a top of the container 30. The opening 33 provides access to the inner cavity 32A of the container 30, for the disposal of waste in the bag A. The opening 33 may be bounded by a hub 34 that is at a top end of the top portion 32. The hub 34 is optionally present, and may be a circular protuberance that projects upwardly from a surface 35 at the top end of the top portion 32. The surface 35 is optional, as webs or the like may support the hub 34. Moreover, the surface 35 could act as a hub or rotational support, such that the circular protuberance may be optional.

A rim 36 may surround the surface 35, whereby an annular compartment 37 surrounds the opening 33. The rim 36 may be a skirt like featuring integrally molded with a remainder of the top portion 32. The rim 36 may for instance have a peripheral shoulder 36A that will collaborate with the lid assembly 50, to form a continuous surface when the lid assembly 40 closes the opening 33. Depressions 36B may also be formed into the wall of the rim 36, and will define pivot portions for the lid assembly 50. The depressions 36B, as pivot(s) can take other forms.

Slots 37A may be circumferentially distributed in the compartment 37, for instance at an intersection between the surface 35 and the wall of the rim 36. The slots 37A are present for the attachment of parts of the bag twisting mechanism 40 to the container 30, in the manner described below. The slots 37A may be located elsewhere, or may be replaced by other attachment features, such as catches, holes, abutments, etc.

Still referring to FIG. 1, the hub 34 is configured to rotatably support the bag twisting mechanism 40, for the bag twisting mechanism 40 to twist the bag closed or open. The bag twisting mechanism 40 has a rotor 41 that is in line with the opening 33. Other expressions that may be used for the rotor 41 are collar, support, neck, among other possible names. The rotor 41 may be the interface between the bag twisting mechanism 40 and the container 30. The rotor 41 is shown having a rotor base 41A that sits on the hub 34 so as to be in sliding contact with the hub 34. Rollers may be present to assist the rolling motion, but as shown, the rotor 41 may be without rollers to rely instead on sliding movement. Consequently, the rotor base 41A is free to rotate relative to the hub 34. The rotor 41 may further include a funnel portion 41B projecting upwardly from the rotor base 41A. The funnel portion 41B defines the central opening of the rotor 41, and flares upwardly from the rotor base 41A. In the embodiment shown, the funnel portion 41B transitions from a circular shape at the rotor base 41A to a non-circular shape at its top periphery 41C, with FIG. 1 showing an exemplary squircle shape. Other shapes are considered as well, including a circular outline at the top periphery, and further including shapes other than a funnel. The non-circular shape may be advantageous in providing purchase points for the film 25.

The top periphery 41C of the funnel portion 41B may serve as a bag hook, i.e., as an attachment for the bag dispensed by the cassette 20. Additional bag hooking features, such as protuberances may be present as well. If the opening 33 is round, the bag hooks may be distributed at 0/90/180/270 degrees, or 0/120/240 degrees, among numerous other examples. As an example, there may be two diametrically opposed elongated bag hooks. However, the bag twisting mechanism 40 may rely on its rotor 41 alone for attachment of the bag, with a top of the bag being folded over the rotor 41 and being retained by friction. The rotor 41 may be a monolithic molded component. Therefore, for the continuous top periphery 41C to act as attachment, its size must be such that the bag end folded over it must be stretched, for it to be retained to the collar 41 by friction.

Still referring to FIG. 1, the bag twisting mechanism 40 may further include a handling ring 42. The handling ring 42 may emulate part of the shape of the funnel portion 41B, so as to sit onto the funnel portion 41B, with the film 25 sandwiched between the handling ring 42 and the funnel portion 41B, in the manner shown in FIGS. 7 to 10. The handling ring 42 may consequently define a central opening that is aligned with the opening 33 in the container 30, and may have a funnel shape as well and as shown, though optional. The handling ring 42 may have a funnel shape while the rotor 41 does not. As the handling ring 42 and the funnel portion 41B are shown as non circular, the handling ring 42 and the rotor 41 cannot rotate relative to one another. Moreover, if the rotor 41 rotates relative to the container 30, the handling ring 42 rotates with the rotor 41. Webs of material 42A may project downwardly from the top peripheral edge of the handing ring 42, to provide additional grip to the bag A hanging from the corners of the rotor 41, i.e., holding the bag captive.

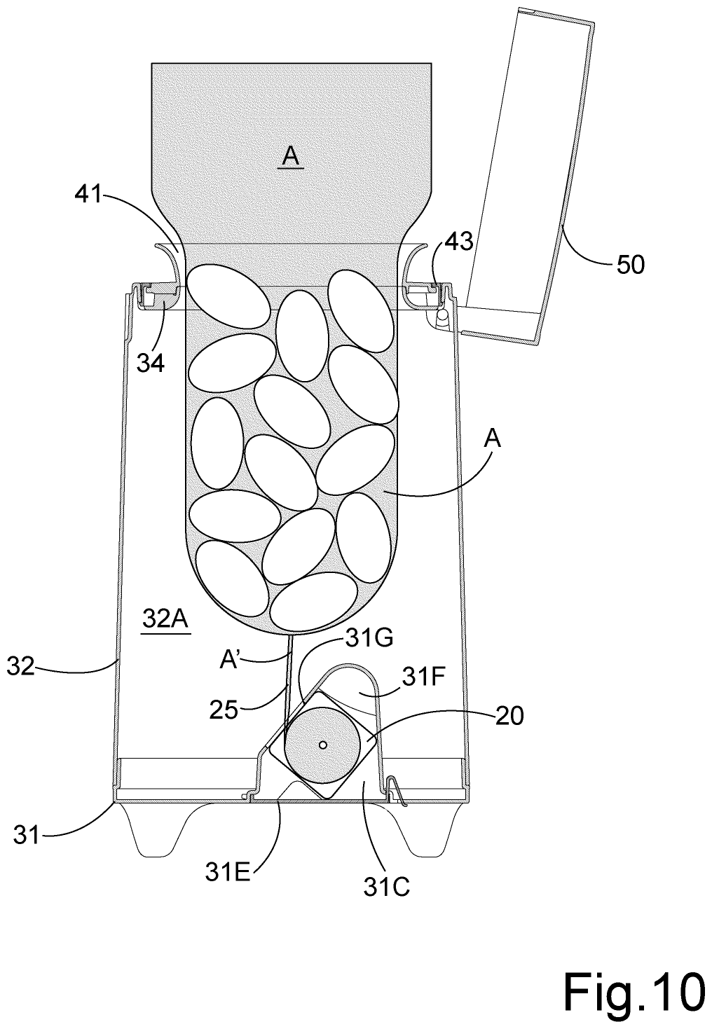

A handle 42B may be connected to the handling ring 42. The handle 42B may project upwardly as shown, or may be in other locations and/or orientations. The handle 42B is readily accessible from a top of the device 10 when the lid assembly 50 is opened. Therefore, a user may grasp the handle 42B and rotate the rotor 41 and handling ring 42 concurrently. In an embodiment, the handling ring 42 is simply laid onto the rotor 41, and can be lifted out of engagement with the rotor 41. It is nonetheless considered to provide locking or latching features as well to releasably lock or attach the handling ring 42 to the rotor 41. In removing the handling ring 42, the top portion of the bag 41 is accessible, for instance to pull a full bag out of the device 10, as in FIG. 10, or to install a top edge of a bag onto the rotor 41.

Still referring to FIGS. 1 and 7-10, the bag twisting mechanism 40 may further include a retaining ring 43, that is also optional. The retaining ring 43 may be present to ensure that the rotor 41 remains in its rotational engagement with the hub 34. In an embodiment, the retaining ring 43 keeps the hub 34 and the rotor 41 in a single rotational degree of freedom engagement. This single rotational degree of freedom may be limited in range, for instance by the presence of an abutment and stops. The limit in range may be present to ensure that the device 10 is used in reciprocating movements between an open bag position and a closed bag position, as described below with reference to FIGS. 7 and 9. The retaining ring 43 may have a peripheral wall 43A with tongues 43B to penetrate the slots 37A in the compartment 37 of the container 30 to be snap-fitted into engagement with the container 30. Other attachment arrangements are considered as well, such as cantilevered fingers, a latch, screws, bolts and nuts, etc. A top annular wall 43C of the retaining ring 43 defines a central opening that is large enough for the base portion 41A of the rotor 41 to pass through it. In an embodiment, the top periphery 41C of the rotor 41 does not pass through the central opening of the retainer ring 43. The top annular wall 43C may sit in a shoulder of the base portion 41A of the rotor 41, in a rotational sliding joint arrangement. Moreover, the arrangement between the top annular wall 43C and the base portion 41A may be such that the rotor 41 is held captive between the retainer ring 43 and the hub 34, but this arrangement is optional.

Therefore, when assembled in the manner shown in FIGS. 7-10, the retaining ring 43 holds the rotor 41 in rotating engagement with the hub 34. The funnel portion 41B of the rotor 41 is above a plane of the top annular wall 43C, for a user to access the top edge of the bag A. The handling ring 42 is also above the plane of the top annular wall 43C, for a user to access the handle 42B, and remove the handling ring 42 to access the top edge of the bag A.

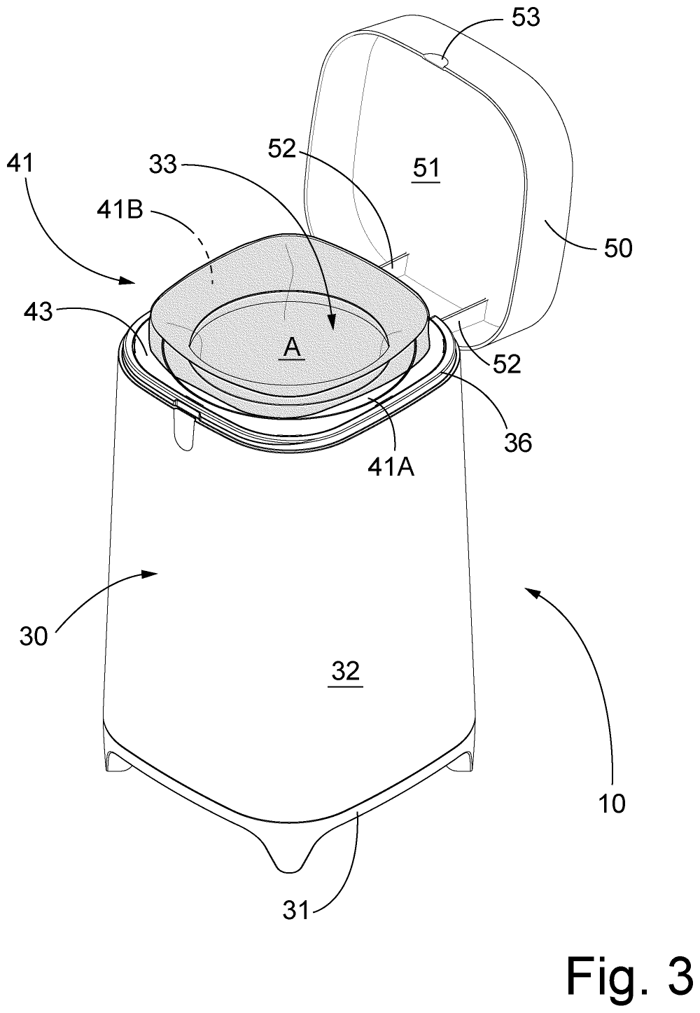

Referring to FIG. 2, the lid assembly 50 has a lid portion 51. The geometry of the lid portion 51 is complementary to that of the upper end of the top portion 32 of the container 30. Accordingly, the lid portion 51 of the lid assembly 50 is seated on the upper end of the container 30, such as on the rim 36. In the illustrated embodiment, the lid portion 51 has pivot members 52 that may be hingedly connected into the depressions 36B so as to pivot, for example from the open orientation of FIG. 2 to a shut orientation. Other pivot arrangements are possible. It is also possible to simply lay the lid assembly 50 onto the container 30, for the lid assembly 50 to be removed when the pail is to be used. Moreover, the lid assembly 50 may have its own lid and base, as opposed to be a lid in and of itself. As shown in FIGS. 2 and 3, a detent 53 may be present to releasably connect to a catch in the top portion 32 of the container 30. Other interconnection arrangements are considered such as a press-fit, quick connect system, threading engagement, or the like. The lid assembly 50 may for instance be pivoted to the open orientation of FIG. 2 to attach or detach a bag to or from the rotor 41 of the bag twisting mechanism 40.

Now that the various components of the device 10 and of the cassette 20 have been described, an operation thereof is explained with reference to FIGS. 4-10. In an embodiment, the film 25 is pulled from a bottom of the container 30, and installed onto any component of the rotor, such as the rotor 41, by a top edge of the film 25 hooked and/or folded over the rotor 41. This may require the temporary removal of the handling ring 42 as in FIG. 3 and/or lid assembly 50, though the lid assembly 50 may be pivoted open as in FIG. 3 if it is present and pivotable. The handling ring 42, if present, may then be reinstalled onto the rotor 41, with the top edge of the bag 25 being held captive. In FIG. 7, with the cassette 20 feeding film 25 from a bottom of the container 30, and with the film 25 attached to the rotor 41, the film 25 is shown in its opened condition, with the lid assembly 50 pivoted open. Accordingly, waste may be disposed into the bag of film 25.

Referring to FIG. 8, when the user has finished with the waste disposal, the user may rotate the bag twisting mechanism 40 by manipulating the handle 42B. As the film 25 is connected to the cassette 20, this causes a twist in the bag of film 25, whereby odors are kept captive in the bag twisted closed. In an alternative embodiment, a motorization unit may automate the rotation, and may do so upon pressing a button or closing the lid assembly 50 (with appropriate sensors present therefor). For example, a bi-directional motor 60 is shown in FIG. 1, along with a gear section 61 on the rotor base 41A. The gear section 61 may be on the full periphery of the rotor base 41A, or on only a portion thereof. If a motorization unit is present, some of the components, such as the handling ring 42 and/or handle 42B may be present or absent. The motorization unit may be actuatable by a button or like interface, and may also include a sensor. For example, a button may be pushed to open the bag via the motor 60. A sensor may then detect that waste has been disposed of, causing a return of the bag to the closed position.

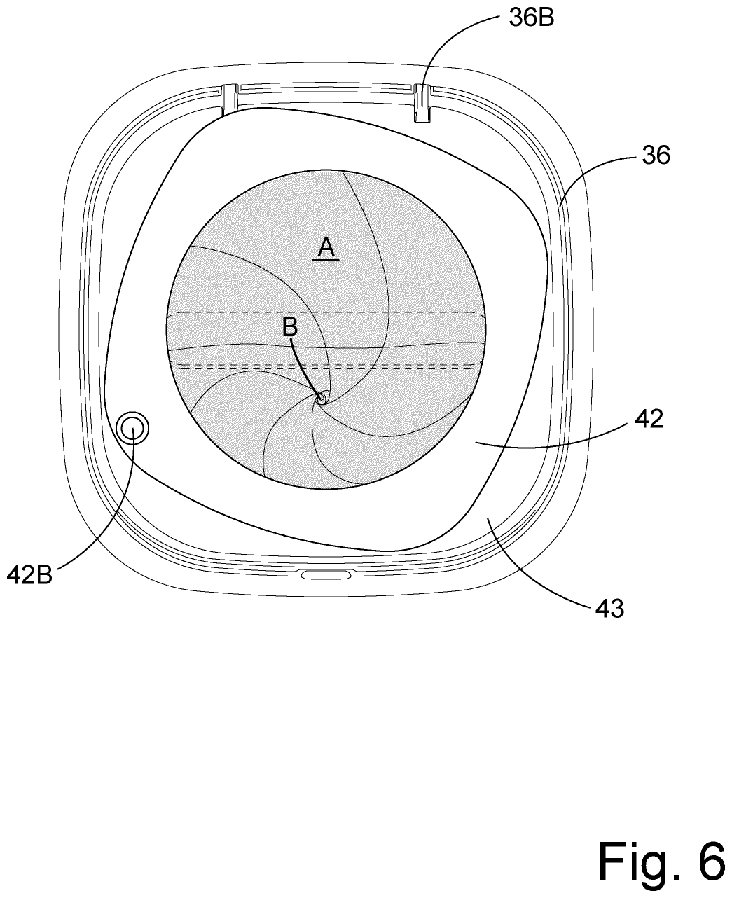

Referring to FIG. 9, the lid assembly 50 may be pivoted shut, with the bag of film 25 twisted shut. As mentioned above, the pivoting of the lid assembly 50 may cause the automated twisting of the bag of film 25. The rotation may be reciprocating between two positions, with the open bag in FIG. 4 and the closed bag in FIG. 6. The rotational may be of less than 90 degrees, as shown (about 75 degrees), though it may be more. The rotation may also be unidirectional, for the bag to form a sausage like bag of waste increments. However, the bi-direction rotation between open and shut conditions reduces the amount of film 25 used, in comparison to a unidirectional rotation capacity.

To reuse the device 10 in a waste disposal operation, the reverse steps may occur. The lid assembly 50 is pivoted away from the arrangement of FIG. 9, to untwist the bag of film 25 and the bag twisting mechanism 40. The bag twisting mechanism 40 is rotated in the opposite direction to open the bag of film 25, i.e., from being twisted shut as in FIG. 8 to being opened as in FIG. 7. Again, such steps may be automated.

Consequently, the user may dispose of waste in the bag of film 25. It is observed that the rotation is reciprocal between the open and twisted shut configurations, such that the bag of film 25 does not feature a twist knot at each waste disposal. The bag of film 25 therefore has more waste per surface area of film 25 than for rotations in a same orientation. In order for the bag of film 25 to twist, it may be required that a bottom of the bag be held. In the illustrated embodiment, the fact that the bag is part of a roll ensures that the bag in use is held at its bottom. It is contemplated to provide other features in the device 10 to hold a bottom of the bag. For instance, a clip, a wire rack, etc may be used to hold the bottom of the bag 25 while the top of the bag 25 is rotated. In a variant, the content of the bag has sufficient weight and/or volume for the bottom of the bag 25 to remain immovable while the top of the bag is twisted.

Once the bag A is full as in FIG. 10, it may be removed from the device 10. To do so, the lid assembly 50, if present, is pivoted open, and the handling ring 42, if present, is removed. The film 25 is manually detached from its hooking onto the rotor 41, and may be pulled upwardly. As the bag A may be part of a roll or series or series of attached bags, the action of pulling the bag A upwardly out of the device 10 causes the next bag, shown as A′ in FIG. 10, to be pulled outwardly, for instance from the cassette 20. The bag A is detached from bag A′, and the bag A′ may be hooked onto the rotor 41. The handling ring 42 may then be reinstalled onto the rotor 41, ready for additional waste to be received in the bag A′.

Referring to FIG. 16, an exemplary bag roll for the bag roll cassette 20 is generally shown at 120. The bag roll 120, is rolled onto a tube 130, and is inserted into a cassette body, such as the cassette body 21 of (FIG. 3). The bag roll 120 in accordance with the present disclosure may also be with or without the tube 130 and/or the cassette body 21 (FIG. 14). The bag roll 120 may be used in the cassette 20, or in any other type of cassette, or by itself. The bag roll 120 is well suited to be used in the device 20, but may be used in other waste disposal devices, such as those described in U.S. patent application Ser. No. 16/603,113, or in U.S. patent application Ser. No. 17/277,361, as examples among others.

The bag roll 120 may be made of a tubular film of plastic material. For example, depending on the contemplated use, the plastic material may be of plastics such as polyethylene (LDPE, LLDPE or HDPE), bioplastics, polylactic acid, to name a few of numerous possible materials. It is also contemplated to add functional layers to the plastic film, such as ethylene vinyl alcohol, for the film to form an odor barrier, or a nylon layer to reinforce the bag. The film may also be biodegradable and/or compostable material, such as starch-based or plant-based materials.

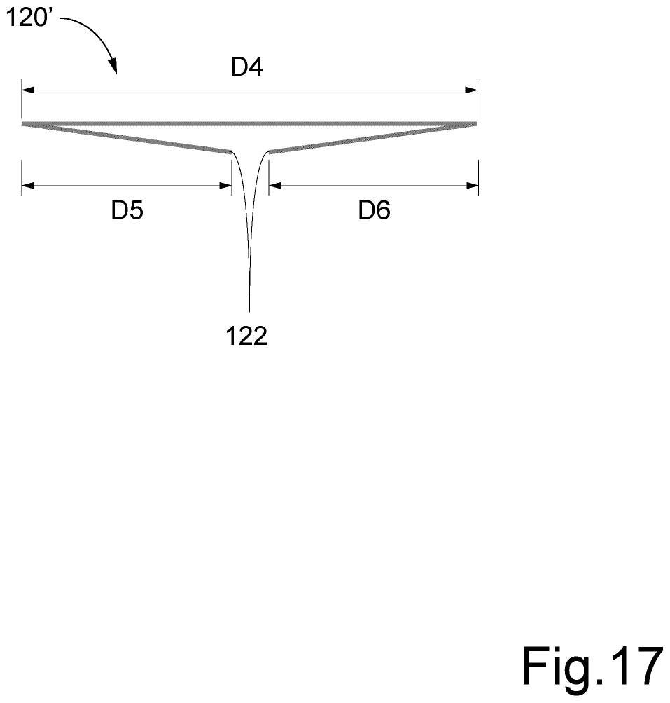

The bag roll 120 may be in the form of a length of tubular film, with weld lines and tear-off perforations delimiting a plurality of bags interconnected end to end, as shown as 120′, 120″ and so on. Any of the bags 120′ may be available on its own and not part of a series or roll of interconnected bags. The tubular film may be accumulated on a roll, in a zig-zag pattern (fanfold), etc. The bag roll 120 may consequently appear to be a continuous sheet as in FIG. 16 due to the thinness of the film, though the bag roll 120 has a pair of panels 121 overlaid onto one another, and joined at side edges 122. In an embodiment, this is referred to as a flattened two-fold condition of the bags: the bags have two folds, i.e., one at each side edge 122, and no gusset. The side edges 122 may be folds as the bag may be made of a tubular film. Stated differently, the bag roll 120 may be a continuous tubular length of film, that is flattened and hence defines the side edges 122. Thus, even though a pair of panels 121 is described, the boundary between the panels 121 may be a fold line, forming the side edges 122. Although the bags in the bag roll 120 could have gussets in accordance with the present disclosure, the transverse dimensions of the bag may be described herein as being in the flattened two-fold condition. This may be construed as meaning that, even if the bag roll 120 has gussets, the dimensions are for the transverse dimensions of the bag as if it were in a flattened two-fold condition. Even though the bags described herein may not be packaged or sold in the flattened two-fold condition (it may have even more folds, e.g., folded on itself as in FIGS. 13 and 14, in a four-fold condition), the flattened two-fold condition is the condition in which the bags are laid flat on a surface so as to have two lateral folds—two side edges—, between the top edge and the bottom edge. FIG. 17 shows a schematic view of the film of the bag roll 120 being in a four-fold arrangement.

Moreover, reference is made to the bag as being unstretched. This may be construed as meaning that the bags are in their initial condition on the bag roll 120 prior to having a user handle the bags prior to installing same on a disposal unit, which may entail some elastic deformation or plastic deformation. The bags in their initial condition on the bag roll 120 may have been plastically prestretched in manufacturing, but in spite of such prestretching their condition on the bag roll 120 is said to be unstretched.

The side edges 122 of each bag extend from a top edge 123 to a bottom edge 124 of each bag, such as bag 120′, with weld seams 125. In an embodiment, the side edges 122 are fold lines (i.e., resulting from a panel folded onto itself), though they could be weld seams. In the end-to-end connection of the bag roll 120, the bags are interconnected such that a bottom edge 124 of a leading bag 120′ is connected to a top edge 123 of a trailing bag 120″ in the bag roll 120, in contrast to bag rolls in which bottom edges or top edges of adjacent bags are interconnected. In order to give the side edges 122 the shape described above, the weld seam 125 may be a continuous seam extending from one of the side edges 122 to the other to connect the panels 121 laterally, or may have segments. This continuous seam 125 includes a portion that defines the bottom closed end of the bags 120′, 120″, etc. Alternatively, the bags may have separate weld seams 125, i.e., the one(s) for the side edges 122 and the one for the bottom closed end. For simplicity, the expression “weld seams 125” is used in the plural, though a single continuous seam 125 may be present, in a U shape for example. The weld seams 125 may be interpreted as weld seam segments of a continuous weld seam 125. The seam 125 may be created by any appropriate welding or adhering method, such as heat welding, etc, whereby reference is made herein as a weld seam, although other types of jointing is contemplated, such as gluing. The weld seam 125 may also connect the panels 121 to define the bottom edge 124, i.e., the closed bottom end of bags of the bag roll 120. The weld seam 125 may also extend to the top edge 123, although in FIG. 16, the weld seam 125 ends at the larger hooking section 120A. In the embodiment of FIG. 16, the straight segments 122A of the larger hooking section 120A are the two folds in the tubular film as described above for the flattened two-fold condition, the folds defining a boundary between the panels 121. Therefore, in accordance with an embodiment, the weld seam 125 is made periodically in a straight continuous tube—a continuous tubular body—in a flattened two-fold condition, and portions of the film laterally outside of the weld seam 125 may be cut out, though they are shown as forming flaps 121A.

When laid flat as in FIG. 16, the bag of the bag roll 120 has a constant width. For example, the side edges 122 are parallel to one another on the full length of the bags from the bag roll 120. However, due to the presence of the weld seams 125, the dimension of the inside of the bag are reduced from the top edge 123 to the bottom edge 124 as described below. On the outside of the seams 125, the bags 120 define flaps 121A that do not form part of the interior of the bag, in that larger objects penetrating the bags 120 cannot fit into the flaps 121A. The side edges 122 are shown as each having an outline that may include a straight segment 122A adjacent to the top edge 123. Then, the weld seams 125 define a tapering segment 122B that may not be present, another straight segment 122C, and/or another tapering segment 122D. The tapering segment 122D extends from the straight segment 122C to the bottom edge 124 of the bags. The segments 122D may be straight lines or curved lines, tapering (i.e., reducing in cross-sectional dimension) from top to bottom. Though the moniker “straight” is used for segment 122C, the side edges may taper, from a maximum dimension of WC, all the way to the bottom edge 124. The expression “straight segment 122C” is used herein for differentiating purposes, in a non-limitative manner. Accordingly, the bags may have four different separate sections, though fewer combinations of separate sections may be present, namely a larger hooking section 120A, a tapering section 120B, a main storage section 120C and a narrower disposal section 120D, from top to bottom of the bags, such as bag 120′. In an embodiment, the bags are without the tapering section 120B and/or the straight segment 122C, and thus transition directly from the larger hooking section 120A to the narrower disposal section 120D. The straight segments 122A, although shown as being parallel to one another, may taper toward the bottom edge 124. Likewise, the straight segments 122C, although shown as being parallel to one another, may taper toward the bottom edge 124. As yet another embodiment, the seams 125 defining the side edges 122 may taper continuously from the top edge 123 to the bottom edge 124, at a continuous angle, or with two different angles, i.e., one for section 120A and another for section 120D, with no section 120B or 120C. In another embodiment, the bag 120′ (whether or not part of the roll 120) has the straight segments 122A parallel or quasi-parallel to one another, to then taper beyond the larger hooking section 120A. For clarity, the width described herein is the flat width as described above, i.e., with the bags of the roll in a flattened two-fold condition with the panels 121 coplanar. The larger hooking section 120A has a width WA that is greater than a width WC of the main storage section 120C, i.e., WA>WC. Widths WA and WC may be constant throughout most or all of the sections 120A and 120C, respectively, if the segments 122A and 122C are parallel as in FIG. 16. If the segments 122A and 122C taper toward the bottom edge 124, the larger hooking section 120A has a minimum width WA that is greater than a maximum width WC of the main storage section 120C. For example, the width ratio is as follows: 0.60 WA≤WC≤0.95 WA. In another embodiment, the width ratio is as follows 0.65 WA≤WC≤0.85 WA. In an embodiment, the segments 122A are parallel to one another, whereas the segments 122C taper toward the bottom edge 124 (and may be mirror images of another, with or without curvature (i.e., they may or may not be arcuate). The minimum width WD of the narrower disposal section 120D is less than the width WC of the main storage section 120C as follows: 0.50 WC≤WD≤0.98 WC. In an embodiment, WA is equal to 30.0 cm±6.0 cm, and has length L equal to 77.0 cm+20.0 cm/−20.0 cm.

The bags, such as bag 120, have a length L that may be segmented as LA, LB, LC and LD, to be representative of the height of sections 120A, 120B, 120C and 120D, respectively. In accordance with an embodiment, LC is at least twice the length of LA, i.e., LC>2 LA. In accordance with another embodiment including the tapering section 120B, LC is at least twice the length of the combination of LA and LB, i.e., LC>2(LA+LB). LA may be separated into LA1 and LA2 if the top edge 123 defines a concavity or convexity as detailed below. LA2 is the portion of the section 120A below such concavity or convexity. In accordance with an embodiment, LC is at least equal to the length of LD, i.e., LC=LD, but may be at least 10% longer, i.e., LC>1.1 LD. In accordance with an embodiment, 0.30 L>LD>0.15 L to minimize the reduction in open diameter. As the bag 120 is filled when coming out of a throat of a waste disposal device 10, as shown in the previous figures, the tapering of the bag 120 to the bottom edge 124 facilitates the removal of the bag from the device 10.

The top edge 123 may have different outlines. In FIG. 1, the outline of the top edge 123 is a straight segment. Other outlines are contemplated such as (A) a concave sine waveform segment, (B) a convex sine waveform segment, (C) a concave triangle waveform segment. As the bags are interconnected end to end in the bag roll 120 with adjacent bags connected bottom to top as described above, the bottom edge 124 has a shape that is complementary to the top edge 123. Stated differently, if the outline of the top edge 123 is a concave sine waveform, the bottom edge 124 is a convex sine waveform, etc.

Tear-off perforation lines 126 are punctured at the junction between the bottom edge 124 of a first bag, the leading bag such as 120′, and the top edge 123 of the subsequent bag in the bag roll 120, the trailing bag such as 120″. The tear-off perforation line 126 may be defined by a series of spaced-apart punctures along an outline emulating that of the bottom edge 123 and the top edge 124. The tear-off perforation line 126 is adjacent to the weld seam 125, and forms a weakened portion of the bag roll 120, causing a separation of one of the bags, such as 120′ in FIGS. 1 and 2, as a result of a tearing action. In an embodiment, the tear-off perforation lines 126 and cut outs at the sides of the weld seams 125 are done by die cutting.

Referring to FIG. 17, a four-fold condition is shown, but with the bag not totally flat to illustrate the four folds, including those of the side edges 122 and those at the ends of D4. D4 is 17.0 cm±4.0 cm, and D5 and D6 may or may not be equal. There may be a gap between the side edges 122 (facing each other as ends of D5 and D6), or an overlap. D5 and D6 may be between 5.0 and 7.5 cm. These dimensions are given as examples among others.

1.-24. (canceled)

25. A bag comprising
a tubular body with a top open end and a bottom closed end,
the tubular body having a larger open diameter at the top open end, the tubular body narrowing into a narrower open diameter adjacent to a bottom closed end, and
the tubular body in a flattened two-fold condition having a pair of side edges extending from a top edge to a bottom edge, the top edge delimiting the top open end, the bottom edge delimiting the bottom closed end,
wherein at least one weld seam causes the narrowing and defines a taper of the open diameter extending to the bottom closed end.

26. The bag according to claim 25, wherein the at least one weld seam includes a pair of segments defining the taper and being mirror images of one another.

27. The bag according to claim 26, wherein the segments are arcuate.

28. The bag according to claim 26, wherein flaps are adjacent to the segments, the flaps excluded from the open diameter.

29. A bag-dispensing cassette comprising:
a roll of bags connected end to end, wherein the bags spiral from a free end to a center of the roll; and
a casing enclosing the roll, the casing having an opening through which an end of the roll passes to exit the casing;
wherein between 2% and 15% of the bags in the roll in the casing in a loosened condition starting from the free end, a remainder of the bags in roll in the casing being in a taut condition.

30. The bag-dispensing cassette according to claim 29, wherein the bags in the roll are according to claim 25, the bags being connected bottom to top.

31. The bag according to claim 25, wherein the bag includes a straight segment at the top open end having a width WA in the flattened two-fold condition, another straight segment having a width WC in the flattened two-fold condition, WA>WC, a tapering segment between the straight segments, wherein the taper is between WC and the bottom closed end.

32. The bag according to claim 31, wherein the taper has a minimum width WD in the flattened two-fold condition, 0.50 WC≤WD≤0.98 WC.

33. The bag according to claim 32, wherein 0.65 WA≤WC≤0.85 WA.

34. The bag according to claim 31, wherein the straight segment having the width WA has a length LA, the other straight segment having the width WC has a length LC, the tapering segment has a length LB, and the taper has a length LD, wherein LC>2(LA+LB).

35. The bag according to claim 25, wherein the bag has a total length L, the taper has a length LD, wherein 0.30 L>LD>0.15 L.

36. A bag-dispensing cassette comprising:
a roll of a plurality of the bag according to claim 25, the bags of the plurality being connected end to end, wherein the bags spiral from a free end to a center of the roll; and
a casing enclosing the roll, the casing having an opening through which an end of the roll passes to exit the casing.

37. The bag-dispensing cassette of claim 36, wherein between 2% and 15% of the bags in the roll in the casing in a loosened condition starting from the free end, a remainder of the bags in roll in the casing being in a taut condition.
